(19) United States
(12) Patent Application Publication
(10) Pub. No.: US 2018/0369480 A1
(43) Pub. Date: Dec. 27, 2018

(54) INJECTION DEVICE FOR INJECTING DOSED AMOUNTS OF A LIQUID THERAPEUTIC AGENT

(71) Applicant: EMPERRA GMBH E-HEALTH TECHNOLOGIES, Potsdam (DE)

(72) Inventors: Markus BENTRUP, Berlin (DE); Janko SCHILDT, Potsdam (DE)

(21) Appl. No.: 16/060,564

(22) PCT Filed: Nov. 17, 2016
(86) PCT No.: PCT/EP2016/078017

§ 371 Date: Jun. 8, 2018

(30) Foreign Application Priority Data
Dec. 9, 2015 (DE) 10 2015 121 409.1

(51) Int. Cl. A61M 5/20; A61M 5/31; G16H 40/63; A61M 5/315

(52) U.S. Cl. CPC A61M 5/20; A61M 5/3146; G16H 40/63; A61M 2205/52; A61M 2205/215; A61M 2205/3553; A61M 5/3155

(57) ABSTRACT

The invention relates to an injection device for injecting dosed amounts of a liquid therapeutic agent, with a receiving device for the therapeutic agent, an applicator device for transferring the therapeutic agent to an application site, a dosing device for transferring the therapeutic agent from the receiving device to the applicator device, a trigger device for activating the dosing device, and a detection device for detecting the applied amount of the therapeutic agent. A sensor arrangement is provided which comprises at least one identification device, which identifies whether the therapeutic agent has reached the application site or a site different than the application site.

### RELATED APPLICATIONS

This application is a Nationalization of PCT Application No. PCT/EP2016/078017, filed Nov. 17, 2016, and entitled “Injection Device for Injecting Dosed Amounts of a Liquid Therapeutic Agent” which claims priority to German Patent Application No. 102015121409.1, filed Dec. 9, 2015, the disclosure of which is incorporated herein by reference.

### BACKGROUND

The invention relates to an injection device for injecting dosed amounts of a liquid therapeutic agent with the features named in the generic term of claim 1.

Many medicines (therapeutic agents) have to be injected into the body. This applies above all for those medicines that are inactivated when given orally or which decrease in their effectiveness to a decisive degree. These medicines include, in particular, proteins, such as insulin; carbohydrates, such as heparin; antibodies; and most vaccines. In most cases, syringes, medication pens or medication pumps are used for injection into the body.

During insulin therapy, in particular during intensified, conventional insulin therapy, and conventional insulin therapy, insulin is not applied in constant quantities. During conventional insulin therapy, insulin is applied at certain times of the day. The daily routine of the patient is oriented to these times. During intensified, conventional insulin therapy, a basic requirement of insulin is provided, often through a slow, long-lasting impact insulin, basal insulin.

At mealtimes, fast-acting insulin is injected. The dose of the fast acting insulin is essentially oriented to the carbohydrates eaten. The dose is therefore specifically selected depending on the external circumstances. These circumstances include the time of day, the range of movement, diet and so on.

Diabetes mellitus can have severe long-term consequences and can lead to physical harm. This can be significantly reduced by customized insulin therapy, preferably an intensified, conventional insulin therapy. A wrong dose can, however, have short-term consequences such as states of hypoglycemia. The best possible customization of the dose to the respective circumstances is therefore particularly desirable.

For this reason, diabetes mellitus patients are instructed to precisely log their living habits and the doses of insulin administered. Such a protocol usually comprises the measured blood sugar level, the quantity of carbohydrates eaten, the insulin dose injected and the date and time. On the basis of the protocol, the doctor treating the patient or the patient themselves can determine or adjust the respective dose. In particular, therefore, a high level of precision regarding the dose of insulin is decisive.

A widely used injection device for injecting dosed quantities of insulin is the so-called insulin pen. Unlike insulin syringes, with the insulin pen an exchangeable medication holder is used. This holder, also known as a carpule or ampule, is delivered in a filled state by the manufacturer and is inserted into the insulin pen before use. When the pen is used, a needle pierces through the seal disc of the ampule and realizes the parenteral injection of the preselected dose when the insulin is applied. An injection and trigger mechanism triggers an injection stroke during injection, which pushes forward a piston or plug in the ampule and leads to the emission of the preselected dose into the target tissue. The mechanism usually consists of a piston rod with a construction length that corresponds to the ampule plug stroke.

Known insulin pens have the outward appearance of a fairly thick ballpoint pen. They comprise a housing in which the ampule containing the insulin is held. The ampule is usually exchangeable. However, arrangements are also known that are formed as disposable pens. The ampules and their content, dimensions and handling are not standardized. An ampule made by one manufacturer cannot therefore usually be inserted into the pen of another manufacturer.

An insulin pen is known from EP 2 414 009 B1 that enables an adapter arrangement for adapting ampules of different dimensions and contents.

A pen comprises a dosing device. The dose required is set on a dosing button. This dose is then injected into the subcutaneous fatty tissue by means of an injection device, which can be with or without a needle. Insulin pens are known in which the set dose is shown with a display on the dosing button instead of a mechanical display. The display is supplied with energy via a voltage source integrated in the insulin pen. The patient can set the dose and note it in their diabetes diary.

Insulin pens are known in which the logging is conducted automatically in a detecting device integrated into the insulin pen. This is connectable to a data processing unit via a data connection that can be wired or wireless. With regard to the structure and function of such an insulin pen, we refer to the disclosure in WO 2013/079644 A1.

Insulin pens are divided into insulin pens that are disposable and those that are reusable. With disposable insulin pens, the ampule and the dosing mechanism forms a pre-fabricated unit from the manufacturer, and are disposed of jointly after the ampule is emptied. A reuse of the dosing mechanism is not intended. Reusable insulin pens present greater challenges for the user. When the ampule is changed, the piston rod must be reset to the start position. Depending on the model, this is achieved by turning or pushing the piston rod, while at the same time activating a special function in the dosing mechanism.

Reusable insulin pens are further subdivided into manual and semi-automated insulin pens. With manual insulin pens, the user presses an injection button with the force of their fingers, and thus determines the duration and flow of the injection. By contrast, with semi-automated insulin pens, a spring is tensioned manually prior to use, which stores the necessary injection energy. During the actual injection procedure, the spring is unlocked by the user.

Before the actual injection, a ventilation of the injection device is required. This ventilation serves on the one hand to ensure that, in fact, only the therapeutic agent (insulin) reaches the desired site of application. On the other hand, the ventilation serves to secure a precise dose of the therapeutic agent to be injected. The user of the injection device is therefore instructed to conduct a manual ventilation of the injection device before each injection. This is achieved by triggering the injection device before the actual injection.

Here, it is disadvantageous that the manual ventilation by the patient can be flawed. If the injection procedures are automatically recorded in a detecting device, it is often unclear whether they refer to an actual injection or the ventilation of the injection device. As a result, the recordings contain errors and the corresponding evaluation of the insulin doses actually injected can be flawed.

DE 696 25 498 T2 discloses an arrangement of electronically controlled injection devices, in which a position sensor is provided. The position sensor issues signals that correspond to the orientation of the device. As a result, the aim is to achieve a situation in which a forward movement of a piston rod is only made possible when the longitudinal axis of the injection device is oriented in a certain direction.

### BRIEF SUMMARY

The object of the invention is to provide an injection device of the generic type, with which it can be recognized in a simple manner whether the triggering of the injection device is an injection or ventilation.

According to the invention, this object is attained by an injection device with the features named in claim 1. Due to the fact that the injection device has a sensor arrangement that comprises at least one proximity sensor by means of which it is detected whether the therapeutic agent has reached the application site or a site that differs from the application site, it is advantageously possible to automatically detect whether an actual injection or a ventilation has occurred. This information is fed to the detecting device of the injection device, so that during logging, it can be taken into account that a ventilation has occurred and not an injection. This increases the quality of the protocols to be evaluated, so that a more precise dose of the therapeutic agent can be recorded or set.

A proximity sensor is regarded as being a sensor that reacts in a non-contact manner, i.e. without direct contact, to an approach. Through integration of such a proximity sensor into the injection device, it can be detected whether, when the injection is triggered, the injection device is located close to a body on which the desired application site is situated, or whether the injection is made into the air. If the injection is made into the air, a ventilation is detected. In cases where the injection occurs when the proximity sensor is triggered, a desired injection of the therapeutic agent is detected.

In a further preferred embodiment of the invention, the proximity sensor can be capacitive proximity sensors, optical proximity sensors or ultrasound proximity sensors. These are all suitable for detecting the approach of the injection device to a human body.

Particularly preferred is an embodiment when the injection device comprises a proximity sensor and a position sensor. As a result, it is advantageously possible to differentiate via the position sensor whether the injection device is in the vertical or horizontal position, and to differentiate via the proximity sensor whether the injection device is sitting on the skin of the person to be injected or is at a distance from them. If both the proximity sensor and the position sensor indicate that the injection device is horizontal and there is an approach to the skin of the person to be injected, a successful injection can be assumed.

The sensitivity of the sensors, in other words the position sensors and the proximity sensors, is here set in such a manner that specific angle deviations from the horizontal or respectively specific minimum distances or respectively maximum distances from the person to be injected are recorded. Overall, an injection device is therefore provided with which it can automatically be reliably detected whether the procedure is a ventilation procedure or an injection procedure when the injection device is triggered.

Further preferred embodiments of the invention result from the other features named in the subclaims.

### BRIEF DESCRIPTION OF THE DRAWINGS

FIG. 1. shows a schematic view of an injection device of the present invention.

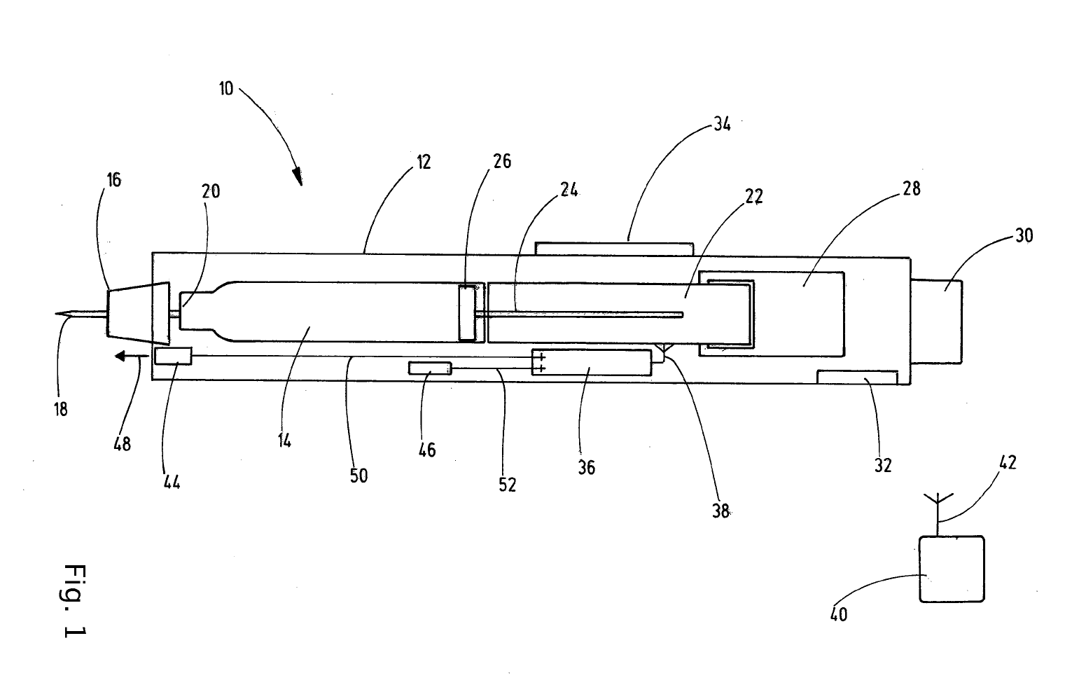

### DETAILED DESCRIPTION

The invention will now be explained in greater detail in an exemplary embodiment with reference to the related drawing, which shows a schematic view of an injection device according to the invention.

The figure shows a schematic view of an injection device which is assigned the numeral 10 overall. The structure and function of injection devices 10 are generally known, so that no further detail is provided within the scope of this description. This can, for example, be an injection device with or without a needle.

The injection device can also be a disposable or a reusable one. Furthermore, the injection device can be equipped with or without an adapter for receiving different ampules from different manufacturers.

The injection device 10 has a housing 12, within which a receiving device 14 is arranged for receiving a therapeutic agent for injection; below, insulin is assumed. The receiving device 14 can be an ampule or respectively a carpule.

The injection device 10 further comprises an applicator device 16 for transferring the insulin to an injection site (application site). The injection site is, for example, an area of skin of a patient. The application device 16 can, for this purpose, have a pin needle 18, which pierces the skin of the patient. The application device 16 is arranged exchangeably on the housing and with the pin 18 pierces a membrane 20 of the receiving device 14.

The injection device 10 further comprises a dosing device 22, which has an actuation element 24, which is in active contact with a plug 26 on the receiving device 14.

The dosing device 22 is assigned a trigger device 28. The trigger device 28 acts together with the dosing device 22.

The injection device 10 further comprises a dosing button 30, via which the dose of insulin to be injected can be set. Further, a trigger button 32 is provided, which is actively connected to the trigger device.

The injection device 10 further comprises a display 34, which is equipped with a display field.

The injection device 10 further comprises a detecting device 36, which is connectable via an interface 38 to a data processing unit 40 that is only implied here. The data processing unit 40 also has an interface 42, which can communicate with the interface 38. The connection can here be wired or wireless.

The injection device 10 further comprises a proximity sensor 44 and a position sensor 46. A detection direction of the proximity sensor 44 lies in the longitudinal extension of the injection device 10, indicated here by an arrow 48.

The position sensor 46 has a sensitive device, such as a correspondingly situated seismic mass, with which it is detected whether the injection device 10 is in a horizontal position, as shown in the figure, or in a vertical position that deviates from this. The seismic mass can be designed in such a manner that the horizontal position is also recognized when there is a deviation of e.g. an angle range of ±30° in relation to a horizontal.

Both the proximity sensor 44 and the position sensor 46 are connected via connection lines 50 or respectively 52 to the detecting device 36. Both the position sensor 46 and the proximity sensor 44 and the connection lines 50 and 52 can be integrated into the housing 12 of the injection device 10, so that they have a defined position in relation to the housing 12.

The injection device 10 represented in the figure shows the following function:

With a correct use of the injection device 10, a ventilation should be conducted prior to the actual injection. This ventilation assists in the secure removal of air located in the pin 18. For this purpose, a lower dose of insulin is injected by the pin 18 into the air.

For a desired injection, the quantity of insulin to be dosed is set using the dosing button 30. The set quantity can be read on the display, and is thus controllable. After setting the injection device 10 with the pin 18 of the applicator device 16 onto the skin of the patient to be treated, the trigger button 32 is actuated. Then, the injection procedure is triggered by means of the trigger device 18, when the actuation element 24 of the dosing device 22 is subjected to a propulsion force. As a result, the actuation element 24 suppresses the plug 26 within the receiving device 14, so that the desired set dose of insulin can be injected via the application device 16. Such a structure and such a function of the injection device 10 are known per se.

By means of the proximity sensor 44, it can be detected during an injection whether an object, here the skin of the person receiving the injection, is located in the direction of injection 48. If this is not the case, the injection is evaluated as ventilation and is registered and recorded accordingly by the detecting device 36.

By means of the position sensor 46, it can additionally be detected whether the injection device 10 is in a typical injection position, namely in an approximately level or respectively horizontal position.

Overall, with the injection device 10 according to the invention, it is achieved that the dosage accuracy is increased. Any ventilations of the injection device 10 are reliably automatically also detected as such and are logged accordingly via the detecting device 36. When the protocols are later evaluated by a doctor treating the patient, the quantity of insulin actually applied can thus be established more precisely.

According to further exemplary embodiments not shown, the injection device 10 can for example also have only the proximity sensor 44 and also only the position sensor 46.

### LIST OF REFERENCE NUMERALS

10 Injection device

12 Housing

14 Receiving device

16 Application device

18 Pin

20 Membrane

22 Dosing device

24 Actuation element

26 Plug

28 Trigger device

30Dosing button

32 Trigger button

34 Display

36 Detecting device

38 Interface

40 Data processing unit

42 Interface

44 Proximity sensor

46 Position sensor

48 Arrow/injection device

50 Connection device

52 Connection line

1. An injection device for injection of dosed quantities of a liquid therapeutic agent, with a receiving device for the therapeutic agent, an applicator device for transferring the therapeutic agent to an application site, a dosing device for transferring the therapeutic agent from the receiving device to the applicator device, a trigger device for activating the dosing device, and a detection device for detecting the applied amount of the therapeutic agent, characterized by a sensor arrangement, which comprises at least one proximity sensor, by means of which it is detected whether the therapeutic agent has reached the application site or a site that differs from the application site.

2. The injection device according to claim 1, characterized in that the proximity sensor is a capacitive sensor and/or an optical sensor and/or an ultrasound sensor.

3. The injection device to claim 1, characterized in that the sensor arrangement comprises a proximity sensor and a position sensor.

4. The injection device according to claim 3, characterized in that the proximity sensor and/or the position sensor are integrated in a housing of the injection device.

5. The injection device according to claim 1, characterized in that the detection device is connectable to a data processing unit.
